(19) United States
(12) Patent Application Publication
(10) Pub. No.: US 2026/0284325 A1
(43) Pub. Date: Sep. 24, 2026

(54) HOLDER FOR MEDICAMENT INJECTOR, MEDICAMENT INJECTION ASSEMBLY, AND METHODS OF USE

(71) Applicant: Purdue Research Foundation, West Lafayette, IN (US)

(72) Inventors: Brian Mark Shepler, West Lafayette, IN (US); Elena Marie Kline, Macomb, MI (US); Jay Jon Kline, Macomb, MI (US); Paula Allison Burge, Martinsville, IN (US); Jacob Alan Flagle, New Palestine, IN (US); Sukesh Jagannathan Ranganathan, West Lafayette, IN (US); Grace Atilola, Indianapolis, IN (US); Akshay Padmanabhuni, Los Angeles, CA (US); Yoon Jae Kim, Seoul (KR)

(21) Appl. No.: 19/572,293

(22) Filed: Mar. 19, 2026

Related U.S. Application Data
(60) Provisional application No. 63/774,418, filed on Mar. 19, 2025.

(51) Int. Cl. A61M 5/32; A61M 5/24

(52) U.S. Cl. CPC A61M 5/3298; A61M 5/24

(57) ABSTRACT

Holders for medicament injectors, medicament injection assemblies, and methods associated therewith. The medicament injection assembly includes the holder, an injector pen, and one or more needles. The holder has a main body with an injector receiver for an injector pen and a needle carousel rotatably coupled to the main body. The needle carousel has a plurality of needle receivers for holding needles and rotates to successively align each needle receiver with the injector receiver. A ratchet assembly allows the needle carousel to only rotate in one direction to advance the needles. An actuator shifts an injector pen in the injector receiver toward an aligned needle receiver in order to mount a needle on to the injector pen and expose the needle tip on the exterior of the holder. Retracting the injector pen retracts the needle back into the needle receiver and disengages the injector pen from the needle.

### CROSS-REFERENCE TO RELATED APPLICATIONS

This application claims priority to provisional U.S. Patent Application No. 63/774,418 filed March 19, 2025, the contents of which are incorporated herein by reference.

### BACKGROUND OF THE INVENTION

The invention generally relates to medicament injectors. The invention particularly relates to holders for medicament injectors with needle changing functionality, medicament injection assemblies including such a holder, as well as components and methods related thereto.

Medicament injectors are used to inject medicaments into a living body, as a nonlimiting example, a human body (sometimes referred to herein as a “person” or “patient”). Typically, such medicament injectors include an injector needle with a lumen (sometimes simply referred to herein as a "needle") fluidically coupled to a reservoir for the medicament and an injector that forces a measured amount of the medicament from the reservoir through the lumen and into the patient. Basic medical syringes are one of the oldest types of modern medicament injectors.

A more recently developed type of self-administered medicament injector is the pen-type injector (sometimes simply referred to herein as an "injector pen"), such as the well-known insulin pen, which is used to self-inject a medicament into the human body. The first insulin pen revolutionized insulin delivery by allowing patients to self-inject a measured dose of insulin quickly and easily without requiring assistance from a medical professional. However, conventional insulin pens require the user/patient to select the correct needle gauge and length, attach needle cartridges to the pen, and dispose of used needles, all of which require a person to manually handle the needle. These designs can provide a complex user experience and may be particularly challenging for users with manual dexterity difficulties because of the need to manually attach, detach, and dispose of the needles. The manual handling of the needles by users can increase the chances of user error, especially for users who struggle with dexterity challenges, vision impairment, and other motor impairments. In addition, the need to change needles after each use can disrupt the ability to administer insulin.

Therefore, it would be desirable to provide a self-administered medicament injector and methods of administering a medicament that address difficulties arising from having to manually attach, detach, and dispose of needles.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, holders for medicament injector pens, medicament injection assemblies, and methods of administering doses of medicament from an injector pen operatively carried in a holder.

According to a nonlimiting aspect, a holder for a medicament injector pen includes a main body with an injector receiver for receiving an injector pen, a needle carousel rotatably coupled to the main body, and an actuator configured to shift the injector pen when operatively disposed in the injector receiver. The needle carousel includes a plurality of needle receivers for holding needles. The needle carousel rotates on the main body to successively align each needle receiver with the injector receiver. The actuator is adapted to shift the injector pen toward an aligned needle receiver to mount a needle in the aligned needle receiver on the injector pen. The actuator shifts the needle mounted on the injector pen out of the needle receiver and exposes the needle on an exterior of the holder.

According to another nonlimiting aspect, a medicament injection assembly includes an injector pen for injecting a medicament into a patient and a holder for the injector pen. The holder includes a main body with an injector receiver for receiving the injector pen, a needle carousel rotatably coupled to the main body, and an actuator that shifts the injector pen. The needle carousel includes a plurality of needle receivers for holding needles. The needle carousel rotates on the main body to successively align each needle receiver with the injector receiver. The actuator shifts the injector pen toward an aligned one of the needle receivers. The actuator operatively mounts a needle in the aligned needle receiver on the injector pen. The actuator shifts the needle mounted on the injector pen out of the needle receiver so as to expose the needle on an exterior of the holder.

According to still another nonlimiting aspect of the invention, a method of administering a dose of medicament from an injector pen operatively carried in a holder is provided. The holder includes a main body with an injector receiver for receiving the injector pen, a needle carousel rotatably coupled to the main body and having a plurality of needle receivers holding needles therein, and an actuator that shifts the injector pen operatively disposed in the injector receiver toward an aligned one or the needle receivers. The needle carousel can be rotated relative to the main body to align a needle disposed in one of the needle receivers with the injector pen. The actuator is engaged to shift the injector pen into engagement with the aligned needle. The actuator is further engaged to operatively mount the aligned needle onto the injector pen. The actuator is also engaged to advance the needle from the needle receiver until exposed on and protruding from an exterior of the holder. The dose of the medicament can then be administered through the needle.

Technical aspects of holders, medicament injection assemblies, and methods as described above preferably include the ability to facilitate the ability of a user to change needles on an injector pen without having to handle each needle directly.

These and other aspects, arrangements, features, and/or technical effects will become apparent upon detailed inspection of the figures and the following description.

### BRIEF DESCRIPTION OF THE DRAWINGS

FIG. 1 is a perspective view of a medicament injection assembly, including an injector pen and a needle operatively disposed in a holder for the injector pen according a nonlimiting embodiment of the invention.

FIG. 2 is an exploded view of the medicament injection assembly represented in FIG. 1.

FIG. 3 is an exploded view of the holder of FIG. 1 without the injector pen or needle(s) .

FIG. 4 is a side elevation view of a main body of the holder of FIG. 1 with hidden surfaces shown in dashed lines.

FIG. 5 is an upper end view of the main body of FIG. 4 with hidden surfaces shown in dashed lines.

FIG. 6 is a cross-sectional view along a central axis of the main body of FIG. 4.

FIG. 7 is a cross-sectional view along an injector receiver in the main body of FIG. 4.

FIG. 8 is a side view of a shaft of the holder of FIG. 1 for rotatably coupling a cap of the holder to the main body of FIG. 4.

FIG. 9 is a top end view of the shaft of FIG. 8.

FIG. 10 is a top end view of a needle carousel of the holder of FIG. 1 with hidden surfaces shown in dashed lines.

FIG. 11 is a cross-sectional view of the needle carousel of FIG. 10 along a central axis thereof.

FIG. 12 is a side elevation view of the cap of FIG. 8 with hidden surfaces shown in dashed lines.

FIG. 13 is a bottom plan view of the cap of FIG. 12.

FIG. 14 is an end view of a slider of the holder of FIG. 1.

FIG. 15 is a side elevation view of the slider of FIG. 14 with hidden surfaces shown in dashed lines.

FIG. 16 is a perspective view of a slider actuator of the holder of FIG. 1.

### DETAILED DESCRIPTION OF THE INVENTION

The intended purpose of the following detailed description of the invention and the phraseology and terminology employed therein is to describe one or more nonlimiting embodiments of the invention, and to describe certain but not all aspects of what is depicted in the drawings, including the embodiment(s) to which the drawings relate. The following detailed description also identifies certain but not all alternatives of the embodiment(s). As nonlimiting examples, the invention encompasses additional or alternative embodiments in which one or more features or aspects shown and/or described as part of a particular embodiment could be eliminated and also encompasses additional or alternative embodiments that combine two or more features or aspects shown and/or described as part of different embodiments. Therefore, the appended claims, and not the detailed description, are intended to particularly point out subject matter regarded to be aspects of the invention, including certain but not necessarily all of the aspects and alternatives described in the detailed description.

Although the invention will be described hereinafter in reference to a medicament injection assembly (medicament injector) 20 and holder 24 thereof that are represented in the drawings as configured to allow a person to self-administer a dose of insulin by way of self-injection with a pen-type medicament injector ("injector pen") 22, it will be appreciated that the teachings of the invention are also more generally applicable to use with self-administered pen-type medicament injectors configured to administer other types of injectable medicaments into human bodies as well as animal bodies. Thus, use of the terms "insulin needle," "insulin injector pen," or other similar insulin-specific terminology is meant to be relative to one possible example of any injectable medicament and is not intended to limit the scope of the invention to only insulin.

To facilitate the description provided below of the embodiment(s) represented in the drawings, relative terms, including but not limited to, "proximal," "distal," "anterior," "posterior," "vertical," "horizontal," "lateral," "front," "rear," "side," "forward," "rearward," "top," "bottom," "upper," "lower," "above," "below," "right," "left," etc., may be used in reference to the orientation of the medicament injection assembly 20, the holder 24 thereof, and/or injector pen 22 during their use and/or as represented in the drawings. All such relative terms are useful to describe the illustrated embodiment(s) but should not be otherwise interpreted as limiting the scope of the invention.

As used herein the terms "a" and "an" to introduce a feature are used as open-ended, inclusive terms to refer to at least one, or one or more of the features, and are not limited to only one such feature unless otherwise expressly indicated. Similarly, use of the term "the" in reference to a feature previously introduced using the term "a" or "an" does not thereafter limit the feature to only a single instance of such feature unless otherwise expressly indicated.

In the nonlimiting embodiment represented in the drawings, the holder 24, the injector pen 22, and one or more needles 26 are preferably assembled in an operative configuration to form the medicament injection assembly 20 for injecting medicament into a body, such as the living body of a person or other animal. The holder 24 preferably includes a main body 30 with one or more needle receivers 40 for receiving an injector pen 22 therein and a needle carousel 32 rotatably coupled to the main body 30. The needle carousel 32 may have a plurality of needle receivers 40 for holding needles 26 therein. The needle receivers 40 are shown in the drawings as being arranged in a circular array. The needle carousel 32 preferably rotates on the main body 30 to successively align each needle receiver 40 with the injector receiver 50. The rotation may be incremental, incremented so as to ensure proper alignment of the needle receivers 40 with the injector receiver 50 and the injector pen 22 therein. The increments may correspond to an angular distance between adjacent ones of the needle receivers 40 to ensure that each incremental advancement of the needle carousel 32 advances the next needle 26 into alignment with the injector pen 22 and/or the injector receiver 50.

A shaft 34 is shown as rotatably coupling the needle carousel 32 to the main body 30. The shaft 34 may define a central (first) axis 38 defined by an axis of rotation of the needle carousel 32. The injector pen 22 preferably shifts in the injector receiver 50 along a second axis 56, which is shown as radially offset from the central axis 38 so that the injector pen 22 is always radially aligned with the circular array of needles 26 regardless of the rotational relation between the needle carousel 32 and the main body 30. The second axis 56 is shown as parallel to the central axis 38.

An actuator 42 is shown as configured to shift the injector pen 22, when operatively disposed in the injector receiver 50, toward an aligned one of the needle receivers 40 in order to operatively mount a needle 26 in the aligned needle receiver 40 on the carousel 32. Further shifting of the injector pen 22 in the same direction preferably shifts the needle 26, now operatively mounted on the injector pen 22, out of the needle receiver 40 so that the needle 26 is exposed on and protrudes from an exterior of the holder 24. The actuator 42 is preferably configured as a slider that slides the injector pen 22, when operatively disposed in the injector receiver 50, along the second axis 56. The actuator 42 preferably includes a carriage 42C inside the injector receiver 50 that shifts the injector pen 22 along the second axis 56. The actuator 42 is shown as coupled to the carriage 42C by a linkage 42B extending through an axial slot 54 through an outer wall of the main body 30 into the injector receiver 50.

A cap 36 is represented as being provided to cover the needle carousel 32 and the needle receivers 40 to prevent the needles 26 from falling out of the needle receivers 40. The cap 36 preferably includes a needle aperture 82 aligned with the injector receiver 50 and with the aligned needle receiver 40. The cap 36 is shown as rotationally fixed relative to the main body 30 so that the aperture 82 is always properly aligned with the injector receiver 50. The needle carousel 32 is shown as disposed between the cap 36 and the main body 30. The needle carousel 32 preferably rotates relative to the cap 36 and the main body 30, while the cap 36 and the main body 30 remain in a fixed rotational configuration relative to each other.

A ratchet mechanism preferably couples the needle carousel 32 and the main body 30 such that the ratchet mechanism allows the needle carousel 32 to rotate in a first rotational direction relative to the main body 30 to advance clean needles 26 into alignment with the injector receiver 50 and/or the injector pen 22, and also prevents the needle carousel 32 from rotating in a second rotational direction opposite the first rotational direction relative to the main body 30. This way, the needle carousel 32 can only be rotated in the direction that advances clean needles 26 for use and prevents previously used needles 26 from being accidentally rotated into use in the opposite direction. The ratchet mechanism preferably causes the carousel 32 to rotate incrementally, for example ,with each rotational increment advancing the next needle receiver 40 into alignment with the injector receiver 50.

The ratcheting mechanism is represented in the drawings as comprising a combination of teeth 72 and pawls 46 on the needle carousel 32 and main body 30 that are adapted to engage each other. The pawl(s) 46 engage the one or more teeth 72 in a manner that allows the needle carousel 32 to rotate in the first rotational direction to advance the clean needles 26 into alignment with the injector pen 22 and to prevent the needle carousel 32 from rotating in the second rotational direction to advance previously used needles 26 into alignment with the injector pen 22.

The medicament injection assembly 20 may be used to administer a dose of medicament from the injector pen 22 when operatively carried in the holder 24. The holder 24 may also be used to automatically mount a clean needle 26 to the injector pen 22 before administering the dose. For example, the needle carousel 32 is preferably rotated relative to the main body 30 to align a clean needle 26 disposed in one of the needle receivers 40 with the injector pen 22. Then, the actuator 42 is preferably engaged to shift the injector pen 22 into engagement with the aligned needle 26 and operatively mount the aligned needle 26 onto the injector pen 22. The actuator 42 may then advance the needle 26 from the needle receiver 40 until the tip of the needle 26 is exposed on and protrudes from the exterior of the holder 24 to thereby be ready for penetrating the skin of a patient. Finally, the dose of the medicament may be administered through the exposed tip of the needle 26. Typically, administration of the dose will be through an automated injector that is part of the injector pen 22 in a customary manner.

The holder 24 may also be used to remove the used needle 26 from the injector pen 22. For example, the actuator 42 is preferably engaged to retract the needle 26 so that the tip of the needle 26 no longer projects from the exterior of the cap 36 and back into the needle receiver 40. Further engagement of the actuator 42 may then dismount the needle 26 from the injector pen 22. The dismounted needle 26 may remain and be stored in the needle receiver 40, thereby eliminating any need for the user to have to deal with disposing of a sharp, used needle 26. Thereafter, before administering another dose, the needle carousel 32 can be rotated an incremented amount to align the next needle receiver 40 containing a fresh, clean needle 26 into alignment with the injector pen 22, and the process may be repeated to administer a new dose using the new, clean needle 26.

When it is desired to change out a used/spent injector pen 22 for a new injector pen 22, the used injector pen 22 may be removed from the injector receiver 50. The injector pen 22 may also be disengaged from the actuator 42. A new injector pen 22 may then be loaded into the injector receiver 50 and operatively engaged with the actuator 42. Thereafter, the new injector pen 22 can be used to administer doses.

When it is desired to provide a new set of needles 26, such as when all of the needles 26 carried in the needle carousel 32 have been used and are dirty, the old carousel 32 may simply be removed from the main body 30 and a new carousel 32 with clean needles 26 loaded in the needle receivers 40 may be mounted to main body 30. The new carousel 32 may come pre-loaded with a set of clean needles 26 to further reduce the need for the user to directly handle any of the needles 26. In some configurations, the shaft 34 and the needle carousel 32, or the shaft 34, the needle carousel 32, and the cap 36, may be provided as a pre-assembled needle carriage assembly for easy replacement. In this configuration, a locking mechanism, such as a locking pin, may be used to releasably lock the shaft 34 to the main body 30. When it is desired to change out the used needles 26 for new needles 26, the locking pin may be removed and/or disengaged from the shaft 34, and the pre-assembled needle carriage assembly may be removed from the main body 30 and disposed of in an appropriate manner. In addition, such a needle carriage assembly may be configured retain the used needles 26 contained inside the needle receivers 40, for example, with the cap 36 or other cover over the needle receivers 40. This may allow the used needles 26 to be disposed of without the user having to directly handle the used needles 26, thereby improving safety for the user. Thereafter, a new pre-assembled needle carriage assembly may be mounted to the main body 30 by inserting its shaft 34 into main body 30 and re-engaging the lock pin with the shaft 34 of the new needle carriage assembly.

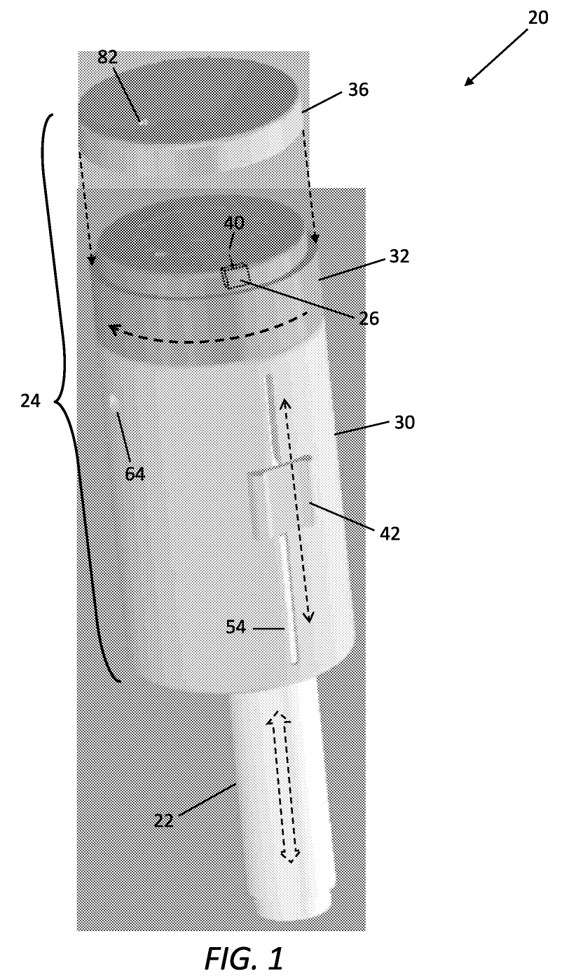

With particular reference to particular figures in the drawings, FIG. 1 shows the illustrated example of the medicament injection assembly 20 as including the injector pen 22 operatively received in the holder 24 for operatively coupling a needle 26 to the injector pen 22 and administering a dose of medicament from the injector pen 22 into a patient. The holder 24 provides an accessory that is preferably able to enhance the process of injection of medicaments, such as for diabetic users that must self-administer an injection of insulin on a regular basis. The holder 24 provides an external needle attachment mechanism for existing insulin pens to automate a needle swap, eliminating the need for the user to remove and/or mount individual needles 26 to a reservoir of the injector pen 22, which in some configurations may provide improved dosage accuracy, safety, and/or ease of use over conventional injection systems.

Because the holder 24 is easily adaptable to operatively receive and function with most typical injector pens, the injector pen 22 may be any suitable type of injector pen having a needle that fluidically couples to a reservoir for the medicament and an injector that forces a measured amount of medicament from the reservoir through the needle into the patient. For example, the injector pen 22 may be a typical insulin pen that holds multiple dosages in the reservoir, has an automated injector that delivers a preselected dosage, and with which the needle 26 is a disposable needle (or needle assembly) that is changed between administration of each dose.

As represented in FIGS. 1-3, the holder 24 includes the main body 30 that receives the injector pen 22 therein, the needle carousel 32 rotatably coupled to the upper end of the main body 30 by the shaft 34, and the cap 36 that covers the upper end of the needle carousel 32. In general, the main body 30 holds the injector pen 22 while the needle carousel 32 rotates relative to the main body 30 to align new needles 26 with the injector pen 22 between each use so that a clean needle 26 can be used with each use. When the clean needle 26 is aligned with the injector pen 22, the injector pen 22 is shifted up toward the needle carousel 32 to mount the clean needle 26 to the injector pen 22, and thereafter the injector pen 22 can be further shifted to push the clean needle 26 through the cap 36 for injecting a dose of the medicament. Thereafter, the injector pen 22 can be retracted inward relative to the cap 36, thereby retracting the now used needle 26 back below the cap 36 and into the needle carousel 32. Further retraction of the injection pen 22 then disengages the injector pen 22 from the used needle 26. When a new dose is to be administered, the needle carousel 32 can simply be rotated to align the next clean needle 26 with the injector pen 22, and the process can be repeated.

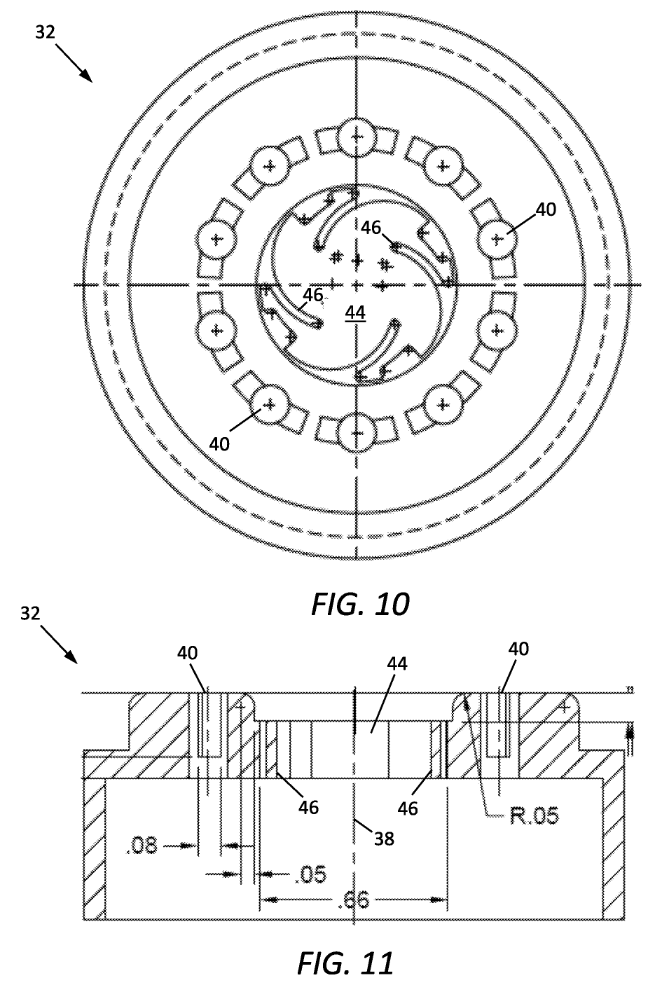

As represented in FIGS. 3, 10, and 11, the needle carousel 32 may be a generally disc-shaped body with an outer peripheral skirt and define a central axis 38 about which the disc-shaped body rotates when operatively mounted to the shaft 34. The needle carousel 32 includes a plurality of needle receivers 40 distributed in a circle around the central axis 38 for holding individual needles 26 for mounting to the injector pen 22. In the represented embodiment, the needle carousel 32 includes ten needle receivers 40 for holding ten different single-use needles 26, one needle 26 disposed in each needle receiver 40. As illustrated in FIG. 1, the needle carousel 32 rotates incrementally in a first direction on the main body 30 to advance the needle receivers 40 and successively align each needle receiver 40 with the injector pen 22 so that the injector pen 22 can be shifted toward the aligned needle receiver 40 by the actuator 42 to automatically operatively mount the needle 26 therein onto the injector pen 22, as described previously and in more detail hereinafter. A central opening 44 through the disc-shaped body of the carousel 32 along the central axis 38 rotatably receives the shaft 34 therethrough. Four pawls 46 are represented as projecting radially inwardly from the peripheral surface of the central opening 44 as part of the ratchet mechanism that allows the needle carousel 32 to rotate in the first rotational direction relative to the main body 30 and prevents the needle carousel 32 from rotating in a second rotational direction opposite the first rotational direction relative to the main body 30. In the represented embodiment, each pawl 46 is formed by a curved flexible finger that extends from a generally tangential convergence with the peripheral surface of the shaft 34 and curves radially inwardly toward the central axis 38. Each needle receiver 40 includes a central shaft that extends completely through the disc-shaped body of the carousel 32 and a pocket on each circumferential side of the central shaft that extends only part way through the disc-shaped body from the top side thereof to form a pair of shoulders on opposite circumferential sides of the central shaft part way down the sides of the central shaft. Thus, a needle 26 can sit on the shoulders while the mounting tip of the injector pen 22 can extend upwardly through the central shaft of the needle receiver 40 to mount the needle 26 thereon. The outer skirt may align with the outer periphery of the main body 30 to provide a comfortable and/or aesthetically pleasing contour between the outer skirt and the main body 30.

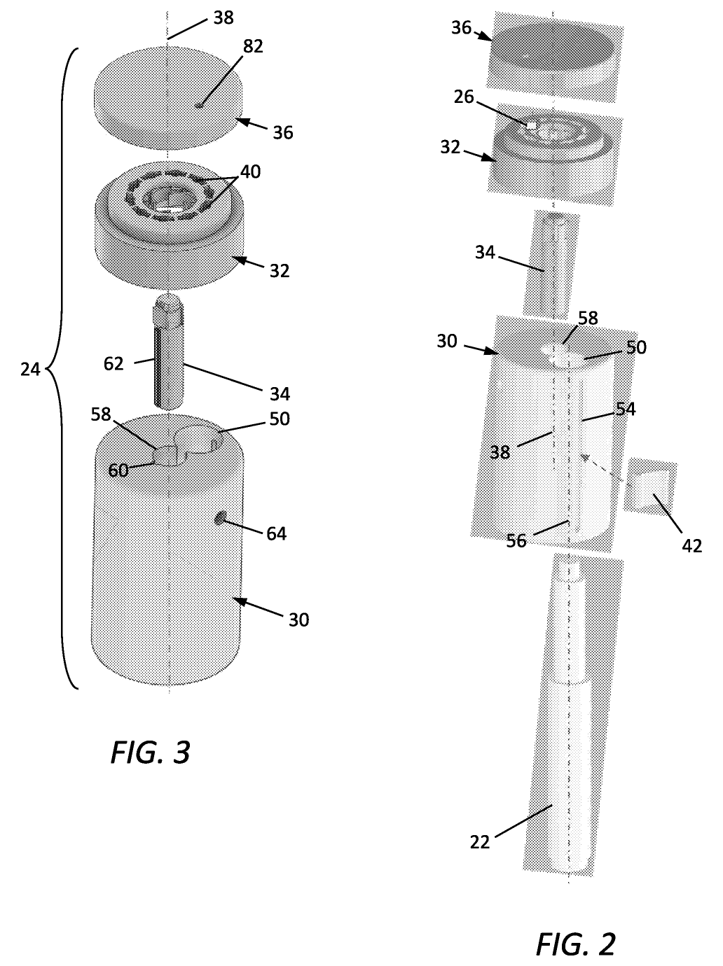

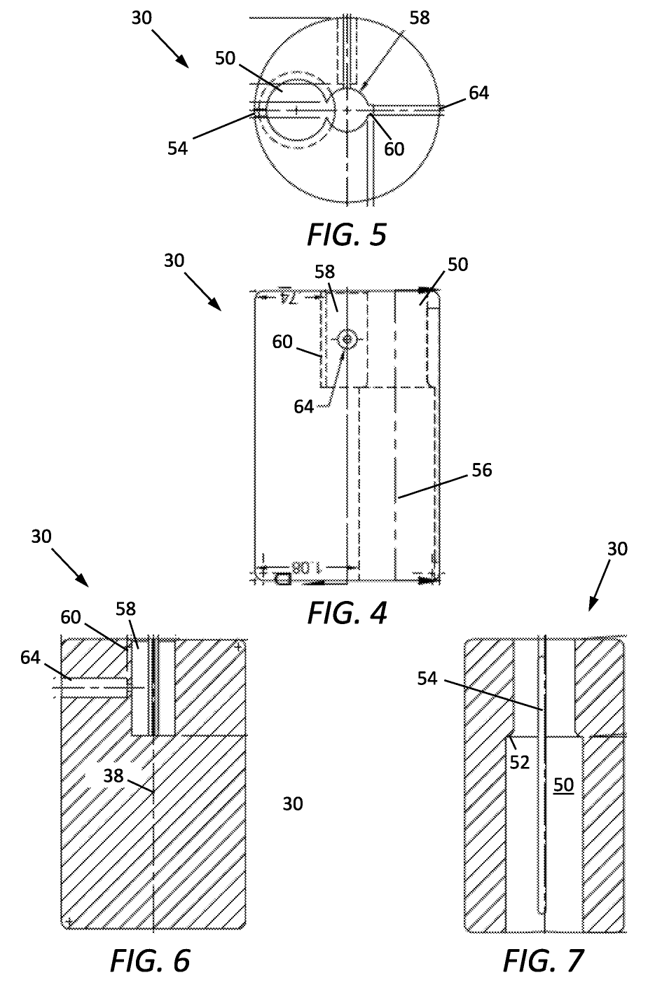

As represented in FIGS. 2 and 5-7, the injector receiver 50 of the main body 30 is defined by a through-bore extending from a lower end of the main body 30 to the upper end of the main body 30. The injector receiver 50 is shaped generally complementary to the outer shape of the injector pen 22 so that the injector pen 22 can be inserted into the lower end of the injector receiver 50. The injector receiver 50 may have a smaller diameter upper end and a larger diameter lower end to prevent the injector pen 22 from sliding all the way through and out of the upper end of the injector receiver 50. The injector receiver 50 may be tapered and/or have a radially stepped or tapered inner peripheral shoulder 52 transition between the smaller diameter upper end and the larger diameter lower end to provide a stop surface for a corresponding surface on the injector pen 22. A slot 54 extends axially along the outer wall of the main body 30 and into the injector receiver 50 for connecting the actuator 42 to the injector pen 22. As represented in FIGS. 14-16, the actuator 42 is in the form of a slider having a finger/thumb engagement pad 42a disposed on an exterior of the main body 30 and a stem 42b projecting out from the back side of the pad 42a connected through the slot 54 to a movable carriage 42C, such as a collar, inside the injector receiver 50. The injector pen 22 releasably couples to the carriage 42C, and movement of the finger engagement pad 42a up and/or down along the slot 54 correspondingly moves the carriage 42C and the injector pen 22 coupled thereto up and/or down along the second axis 56 defined by the through-bore of the injector receiver 50. The second axis 56 is offset radially from the central axis 38 so that the injector pen 22 is radially aligned with the circular array of needle receivers 40 in the needle carousel 32. A second bore 58 extends into the upper end of the main body 30 for receiving the shaft 34. The second bore 58 is represented as a blind bore that includes an axial keyway 60 that engages a corresponding key 62 on the shaft 34 to prevent the shaft 34 from rotating relative to the main body 30. A pin hole 64 extends through the sidewall of the main body 30 into the second bore 58 to receive a pin capable of securing the shaft 34 in the second bore 58, which also locks the needle carousel 32 in its operative position at the upper end of the main body 30.

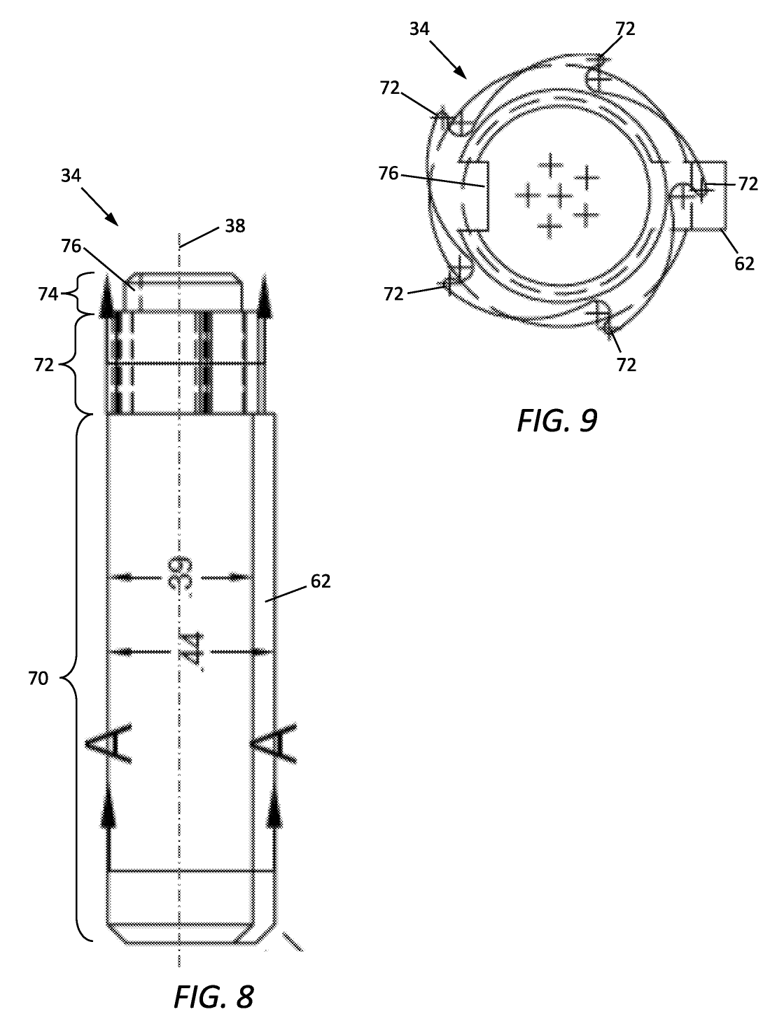

As represented in FIGS. 2, 8, and 9, the shaft 34 includes a generally cylindrical lower portion 70, a set of radial teeth 72 projecting radially outwardly and from a complementary portion of the ratchet mechanism above the lower portion 70, and a top end portion 74 above the teeth 72. The key 62 extends axially along the length of the lower portion 70. The radial teeth 72 are curved so as to interact with the pawls 46 of the needle carousel 32 to rotate incrementally in the advancement direction. The radial teeth 72 and pawls 46 are preferably configured such that each rotational increment advances the needle carousel 32 the angular amount needed to move from one needle receiver 40 to the next adjacent needle receiver 40 into alignment with the injector receiver 50, and thus also the injector pen 22 received therein. Thus, each increment advances the needle carousel 32 the distance between two adjacent needle receivers 40. A second keyway 76 extends axially along the sidewall of the top end portion 74 to interlock with a complementary key 78 on the cap 36. The lower portion 70 of the shaft 34 slides into the second bore 58 with the key 62 disposed in the keyway 60 to prevent the shaft 34 from rotating relative to the main body 30. The radial teeth 72 are disposed above the upper end surface of the main body 30. The needle carousel 32 mounts onto the shaft 34 such that the shaft 34 projects through the central opening 44 with the radial teeth 72 engaged with pawls 46 to provide the ratchet mechanism. The outer skirt of the needle carousel 32 may rest on the upper end of the main body 30.

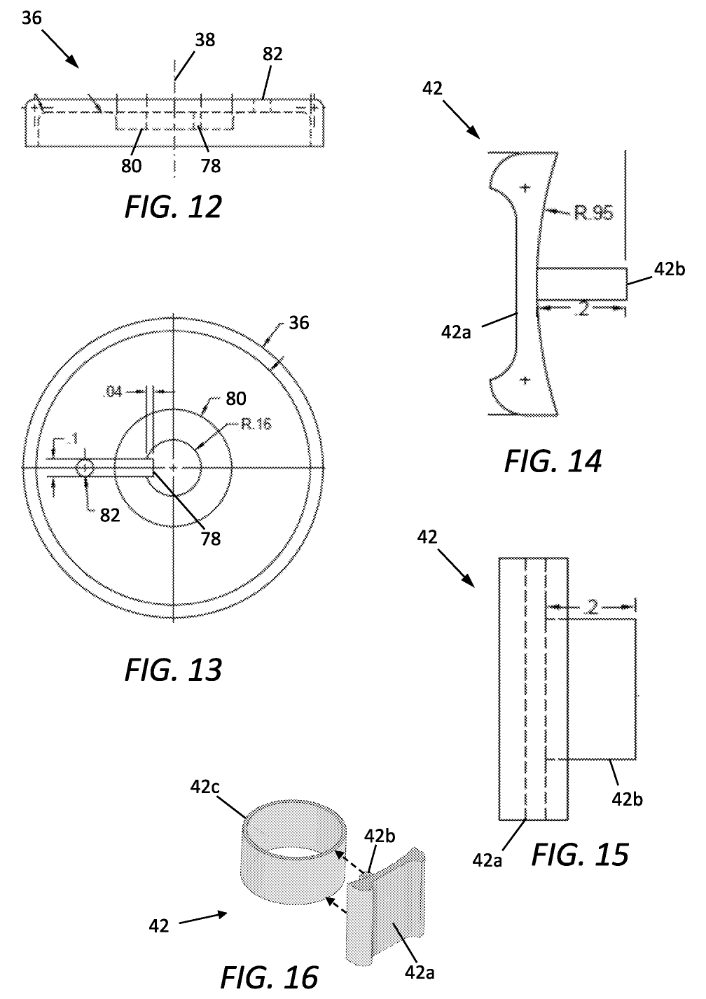

As represented in FIGS. 1-3, the cap 36 mounts onto the top end portion 74 of the shaft 34. As represented in FIGS. 12 and 13, the cap 36 is generally disc-shaped with a peripheral skirt extending downwardly from the outer periphery of its disc-shape. A central mounting collar 80 projects downwardly from the bottom side of the disc-shape and mounts onto the top end portion 75 of the shaft 34. The key 78 projects radially inwardly into the mounting collar 80 to engage the keyway 76 of the shaft 34 and thereby prevent the cap 36 from rotating on the shaft 34 and relative to the main body 30. The needle aperture 82 extends through the cap 36 to allow the needle 26 to be pushed through and protrude from the cap 36 and expose the tip of the needle 26 on the exterior of the cap 36. The needle aperture 82 is axially aligned with the injector receiver 50 and is radially aligned with the circular array of the needle receivers 40. In this way, the needle carousel 32 can be rotated relative to both the main body 30 and the cap 36, while the needle aperture 82 remains axially aligned with the injector receiver 50.

As previously noted above, though the foregoing detailed description describes certain aspects of one or more particular embodiments of the invention, alternatives could be adopted by one skilled in the art. For example, the medicament injection assembly 20, holder 24, and/or their components could differ in appearance and construction from the embodiments described herein and shown in the drawings, functions of certain components of the medicament injection assembly 20 and/or the holder 24 could be performed by components of different construction but capable of a similar (though not necessarily equivalent) function, and various materials could be used in the fabrication of the medicament injection assembly 20, holder 24, and/or their components. As such, and again as was previously noted, it should be understood that the invention is not necessarily limited to any particular embodiment described herein or illustrated in the drawings.

1. A holder for a medicament injector pen, the holder comprising:
a main body with an injector receiver for receiving an injector pen therein;
a needle carousel rotatably coupled to the main body, the needle carousel comprising a plurality of needle receivers for holding needles therein, wherein the needle carousel rotates on the main body to successively align each needle receiver with the injector receiver; and
an actuator configured to shift the injector pen, when operatively disposed in the injector receiver, toward an aligned needle receiver to mount a needle in the aligned needle receiver on the injector pen and to shift the needle mounted on the injector pen out of the needle receiver and expose the needle on an exterior of the holder.

2. The holder for a medicament injector pen of claim 1, further comprising a cap covering the needle carousel and the needle receivers, wherein the cap comprises an aperture aligned with the injector receiver, wherein the needle carousel is disposed between the cap and the main body, wherein the cap does not rotate relative to the main body, and wherein the needle carousel rotates relative to the cap.

3. The holder for a medicament injector pen of claim 1, further comprising a ratchet mechanism coupling the needle carousel and the main body, wherein the ratchet mechanism allows the needle carousel to rotate in a first rotational direction relative to the main body and prevents the needle carousel from rotating in a second rotational direction opposite the first rotational direction relative to the main body.

4. The holder for a medicament injector pen of claim 3, wherein the ratchet mechanism causes the carousel to rotate incrementally, with each rotational increment advancing the next needle receiver into alignment with the injector receiver.

5. The holder for a medicament injector pen of claim 3, a shaft rotatably coupling the needle carousel to the main body, wherein the ratcheting mechanism comprises a plurality of teeth on one of the needle carousel and the main body and at least one pawl on the other of the needle carousel and the main body to engage the teeth to allow the needle carousel to rotate in the first rotational direction and prevent the needle carousel from rotating in the second rotational direction.

6. The holder for a medicament injector pen of claim 5, wherein the shaft defines a first axis about which the needle carousel rotates, and the actuator shifts the injector, when operatively disposed in the injector receiver, along a second axis parallel to the first axis.

7. The holder for a medicament injector pen of claim 1, wherein a first axis is defined by an axis of rotation of the needle carousel, and the actuator comprises a slider that slides the injector pen, when operatively disposed in the injector receiver, along a second axis parallel to the first axis.

8. A medicament injection assembly comprising:
an injector pen for injecting a medicament into a patient; and
a holder for the injector pen, wherein the holder comprises:
a main body with an injector receiver for receiving the injector pen therein;
a needle carousel rotatably coupled to the main body, the needle carousel comprising a plurality of needle receivers for holding needles therein, wherein the needle carousel rotates on the main body to successively align each needle receiver with the injector receiver; and
an actuator that shifts the injector pen toward an aligned one or the needle receivers, operatively mounts a needle in the aligned needle receiver on the injector pen, and shifts the needle mounted on the injector pen out of the needle receiver so as to expose the needle on an exterior of the holder.

9. The medicament injection assembly of claim 8, further comprising a cap covering the needle carousel and the needle receivers, wherein the cap comprises an aperture aligned with the injector receiver, wherein the needle carousel is disposed between the cap and the main body, wherein the cap does not rotate relative to the main body, and wherein the needle carousel rotates relative to the cap to selectively advance needles in the needle receivers into successive alignment with the injector pen.

10. The medicament injection assembly of claim 8, further comprising a ratchet mechanism coupling the needle carousel and the main body, wherein the ratchet mechanism allows the needle carousel to rotate in a first rotational direction relative to the main body and prevents the needle carousel from rotating in a second rotational direction opposite the first rotational direction relative to the main body.

11. The medicament injection assembly of claim 10, wherein the ratchet mechanism causes the carousel to rotate incrementally in succession through the needle receivers such that each rotational increment advances the next needle receiver into alignment with the injector receiver.

12. The medicament injection assembly of claim 10, further comprising a shaft rotatably coupling the needle carousel to the main body, wherein the ratcheting mechanism comprises a plurality of teeth on one of the needle carousel and the main body and at least one pawl on the other of the needle carousel and the main body to engage the teeth, wherein the engagement between the at least one pawl and the teeth allows the needle carousel to rotate in the first rotational direction and prevent the needle carousel from rotating in the second rotational direction.

13. The medicament injection assembly of claim 12, wherein the shaft defines a first axis about which the needle carousel rotates, and the actuator shifts the injector pen along a second axis parallel to the first axis.

14. The holder for a medicament injector of claim 8, wherein a first axis is defined by an axis of rotation of the needle carousel, and the actuator comprises a slider that slides the injector pen along a second axis parallel to the first axis.

15. A method of administering a dose of medicament from an injector pen operatively carried in a holder, wherein the holder comprises a main body with an injector receiver for receiving the injector pen, a needle carousel rotatably coupled to the main body and having a plurality of needle receivers holding needles therein, and an actuator that shifts the injector pen operatively disposed in the injector receiver toward an aligned one or the needle receivers, the method comprising:
rotating the needle carousel relative to the main body to align a needle disposed in one of the needle receivers with the injector pen;
engaging the actuator to shift the injector pen into engagement with the aligned needle, operatively mount the aligned needle onto the injector pen, and advance the needle from the needle receiver until exposed on and protruding from an exterior of the holder; and
administering the dose of the medicament through the needle.

16. The method of claim 15, further comprising engaging the actuator to retract the needle back into the needle receiver and dismount the needle from the injector pen.

17. The method of claim 16, further comprising rotating the needle carousel an incremented amount to align the next needle receiver containing a second needle into alignment with the injector pen.
